(19) United States
(12) Patent Application Publication
(10) Pub. No.: US 2006/0275332 A1
(43) Pub. Date: Dec. 7, 2006

(54) Cosmetic composition

(75) Inventors: Rupesh Kumar Agarwal, Mumbai (IN); Jayashree Anand, Mumbai (IN); Mohini Anand Bapat, Mumbai (IN); Rajan Raghavachari, Mumbai (IN); Ritu Verma, Singapore (SG)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco Inc. d/b/a UNILEVER

(21) Appl. No.: 11/440,951

(22) Filed: May 25, 2006

(30) Foreign Application Priority Data
May 27, 2005 (IN) 0635/MUM/2005
Nov. 29, 2005 (EP) 05257326.8

(51) Int. Cl. A61K 8/02

(52) U.S. Cl. 424/401

(57) ABSTRACT

A topical multiphase cosmetic composition is provided comprising co-extensive emulsion and gel phases. The phases have at least one common interface and are disposed such that the gel phase forms greater than 50% of the outer surface of the composition. The composition provides a combination of the unique sensory and delivery properties of gel and emulsion based cosmetics and allows for their contact with the skin at selected time intervals during application.

### TECHNICAL FIELD OF THE INVENTION

This invention relates to improved cosmetic compositions. More particularly, this invention relates to improved multiphase topical cosmetic compositions.

### BACKGROUND TO THE INVENTION

Cosmetic compositions have long been known in the art. These formulations have always been highly dynamic to meet the ever-changing needs of human beings and also due to the discovery of new and more potent active agents. Cosmetic formulators have therefore been constantly facing increasing need to be innovative and thereby deliver better products to meet the above requirements. One such requirement has been delivery of more than one active through a single composition. Formulators have adopted many approaches in the past to meet this requirement.

In formulations where there is a probability of unfavorable interaction between two or more ingredients an approach has been to encapsulate one of the ingredients in a suitable matrix such as polysaccharides, proteins and gums. The matrix material acts as a physical barrier between ingredients thereby preventing undesirable interactions between them.

In another approach, two or more different formulations each containing one or more active ingredients, are provided in spatially separated tubes or packages such that one formulation does not come in physical contact with the other, thereby preventing undesirable interactions between the ingredients, so long as the formulations are present inside the tube or package.

A known disadvantage of spatially separated tubes is that if the user inadvertently applies unequal force to the package in a view to extrude the product, one composition gets extruded in excess of the other.

In addition to the above requirements a highly desirable feature of cosmetics has been to provide superior visual appearance and aesthetic appeal to attract potential consumers. Cosmetic formulators have achieved this by incorporating two or more visually distinct phases in the composition. This can be achieved by selectively formulating such products, which requires high level of skill. Alternatively and more easily, such products can also be provided in spatially separated tubes/packages as described above. Frequently such packages are transparent thereby allowing for customers to see-through the package into the product.

Furthermore, apart from having an attractive external visual appearance that adds to its consumer acceptability, a cosmetic composition frequently is also intended to deliver various cosmetically active ingredients such as skin lightening agents, antiperspirants, sebum reducing agents, moisturizing agents, anti acne agents, oil-control actives and others. Effective delivery of these actives depends largely on other matrix or base ingredients of the formulation. Therefore, all the above class of active ingredients cannot be delivered through a single universal cosmetic base composition.

Emulsion-based compositions are capable of delivering large number of benefit agents. Consumers also prefer emulsion-based cosmetics as they produce matty and draggy feel and they impart a soft sheen or gloss to the skin. Emulsion based compositions, however are not capable of providing intense localized cooling and moisturization, which is characteristic of hydrophilic gels. Hydrophobic gels are capable of delivering higher amounts of oil-soluble actives and hydrophilic gels are capable of delivering higher amounts of water-soluble actives, when compared with corresponding delivery of actives through oil-based and water-based emulsions. If the user contacts emulsion based cosmetics before the gel-based one in mixed media compositions, the highly desired sensory benefits of gels are liable to be masked by the presence of the emulsion on the skin. Therefore, we have recognised that there is a need to provide a cosmetic composition that allows the user to first experience the desirable sensory properties of a gel but which also provides the desirable characteristics of an emulsion-based cosmetic.

A further problem that is often faced by formulators when attempting to provide two different types of formulations through a single cosmetic composition is that there is a tendency of ingredients to migrate from one phase to the other. This phenomenon is commonly referred to as “bleeding” of ingredients. This happens when the ingredient has a similar affinity for both phases. The level of “bleeding” is largely dependent on the nature of individual phases, i.e, hydrophilic or hydrophobic. Thus this problem is not so acute when one of the phases is hydrophilic and the other phase is hydrophobic, as generally the two phases are incompatible and tend to remain distinct from each other. However, when both the phases are either hydrophilic or hydrophobic, their inherent tendency to blend into each other is high. In addition, in such cases the ingredients contained in one phase have equal affinity for the adjacent phase, which leads to bleeding of the ingredients into the adjacent phase.

U.S. Pat. No. 4,335,103 and U.S. Pat. No. 4,159,028 (both assigned to Almay Inc) disclose a duophase cosmetic cleansing cream composition including two generally separate and stable cosmetic compositions intimately mixed to yield a cleansing type composition that may be applied to a subject's face or body in one step. The composition includes a first cleansing cream phase composition and a gel phase composition. The respective cream and gel phases are intimately blended, mixed, or combined in preferably a swirl or marble like type configuration such that the respective phases exist throughout the entire composition as formed by the two combined phases and wherein each respective phase remains generally separate, stable and visually distinct. Thus the two phases occur in the composition such that the user's skin is likely to simultaneously contact an adequate amount of each of them and so these patents do not describe compositions where a particular phase can be preferentially contacted with the skin while still maintaining the two phases distinct and without intimate blending.

Patent applications CA 2,246,316 and WO98/24399 (both in the name of Henkel KgA) disclose two-phase skin care agents, specially two-phase skin creams, comprising two separated plastic flowing constituents packed in a tube which can be pressed out of the tube in the form of parallel, concentric or striped strips. Said agents can be advantageously produced when the first component has a continuous aqueous phase and the second component has a continuous oil phase. Preferably, both phases are differently colored or pigmented. Preferably, an aqueous gel or an oil in water emulsion are used as a continuous aqueous phase, and a thickened oil or water-in-oil emulsion is used as a continuous oil phase. However, these documents do not disclose a composition wherein the components are disposed such that a gel phase forms greater than 50% of the outer surface of the composition. Furthermore, these documents do not teach a multiphase cosmetic composition where both the components are either hydrophilic or both are hydrophobic.

Patent application WO 2004/100913 (in the name of Unilever) describes multiphase toothpaste compositions comprising an opaque phase and a transparent gel phase, each said phase having a mobile phase with an effective amount of cleansing or polishing agents suspended therein, and wherein of the ingredients making up more than 2% of each mobile phase a corresponding amount of said ingredients is present in the other mobile phase such that no substantial transfer of ingredients occurs between the mobile phases. The toothpaste formulations described are substantially different from topical cosmetic compositions. In particular, the phases described in WO 2004/100913 are simple dispersions of solids in liquids and are unlike cosmetic compositions wherein at least one phase is typically an emulsion.

Thus the prior art has not provided the user with a cosmetic composition that combines the sensory and active-delivery benefits of gel and emulsion based cosmetics, in a single application, wherein the composition is so configured that there is flexibility in providing different sensory properties at different time periods after application of the composition on the substrate.

The present inventors have advantageously combined the benefits of two visually distinct phases, each phase capable of containing and delivering to the substrate one or more of benefit agents, while overcoming the general drawbacks, disadvantages and limitations of such prior art compositions. This has been achieved while maintaining them visually distinct, without the use of spatially separated tubes and while ensuring that on application, substantial portion of gel-based cosmetic comes in contact with the skin of the user before the emulsion phase, thus providing the desired sensory properties.

It is therefore, an object of the invention to provide a multiphase topical cosmetic composition comprising a gel phase and an emulsion phase having good visual appeal.

Another object of the present invention is to provide a multiphase topical cosmetic composition comprising a gel phase and an emulsion phase wherein both the phases deliver different active ingredients in a single application.

Yet further object of the present invention is to provide a multiphase topical cosmetic composition comprising a gel phase and an emulsion phase wherein the composition provides a combination of unique sensory properties of gel and emulsion based cosmetics and allows for their delivery onto the skin at different points in time on application.

### SUMMARY OF THE INVENTION

The present invention provides a topical multiphase cosmetic composition comprising co-extensive emulsion and gel phases wherein the phases have at least one common interface and are disposed such that the gel phase forms greater than 50% of the outer surface of the composition.

Preferably the emulsion phase is an oil-in-water emulsion and the gel phase is a hydrophilic gel.

It is further preferred that the emulsion phase is disposed within the gel phase.

In a most preferred embodiment the emulsion phase is disposed coaxially within the gel phase.

### BRIEF DESCRIPTION OF THE DRAWINGS

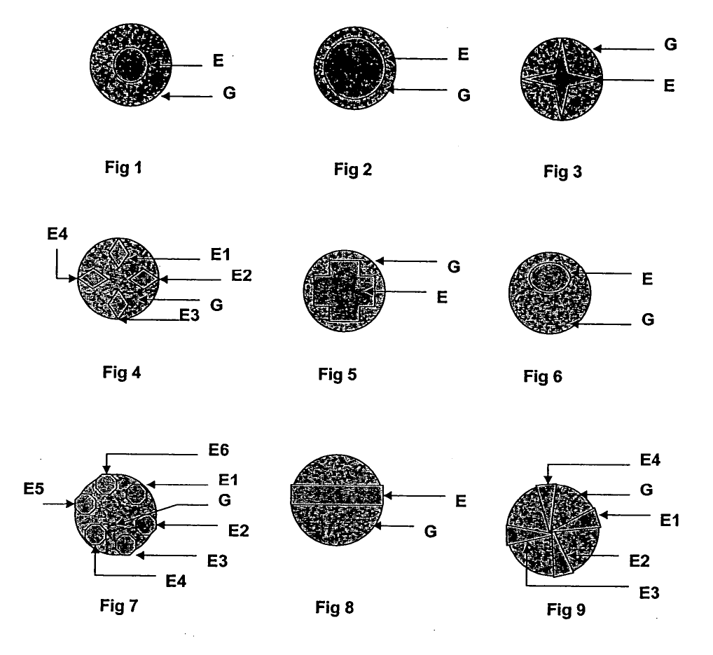

The invention will now be described in detail with reference to the drawings in which FIGS. 1 to 9 show a transverse cross-sectional view of each of nine different embodiments of the cosmetic composition of the invention on extrusion from a tube or package having a dispensing nozzle of circular cross section.

### DETAILED DESCRIPTION OF THE INVENTION

The present invention relates to a topical multiphase cosmetic composition comprising a gel phase and an emulsion phase wherein the composition provides the beneficial effects of both phases in a single application.

Cosmetic Composition

As used herein, the term “outer surface” of the composition means the outer surface of the composition as present in the package and that is exposed to the atmosphere and visible to the human eye when the composition is dispensed from the nozzle/tip of the container. It is to be understood that this outer surface is a function of the shape and geometry of the dispensing tip/nozzle of the container, which is generally circular or square. This shape and geometry has no bearing on the invention. It is also to be understood that the external appearance of the composition after dispensing by extrusion from such containers is similar to the bulk of the composition filled in the container.

The term “co-extensive” means that the two phases extend over the entire length of the container and are capable of being extruded uniformly and simultaneously from the container and can be applied to the substrate in a single application.

In order to enhance the effectiveness of the composition in providing that the user first experiences the effect of the gel phase to a substantial extent before the effect of the emulsion phase, it is preferred that the gel phase forms greater than 80% of the outer surface of the composition. It is further preferred that the gel phase forms the entire outer surface of the composition. More preferably the emulsion phase is coaxially disposed within the gel phase, i.e. the longitudinal axis of emulsion phase is the longitudinal axis of the gel phase when the composition is dispensed from the container and also when the composition is stored within the container. Alternatively or additionally, a preferred aspect provides for the longitudinal axis of the emulsion phase to be parallel to the longitudinal axis of the gel phase.

The emulsion and gel phases can be present in a percentage volume ratio of 10:90 to 90:10, preferably from 30:70 to 70:30 and most preferably from 40:60 to 60:40. In a preferred embodiment, the emulsion and gel phase each form 50% by volume of the composition.

It is preferred that the composition has an oil-in-water emulsion constituting the emulsion phase and a hydrophilic gel forming the gel phase. In an alternative embodiment the emulsion phase is a water-in-oil emulsion and the gel phase is a hydrophobic gel.

The term “multiphase” means that the composition has at least two different phases, an emulsion phase and a gel phase.

The emulsion and gel phases can be disposed in various spatial geometric patterns in the composition, while ensuring that the phases have at least one common interface and the gel phase forms greater than 50% of the outer surface of the composition. Non-limiting examples of such patterns are illustrated in the drawings.

FIGS. 1 to 9 represent the transverse cross sectional view of the composition of the invention on extrusion from a tube or package having a dispensing nozzle of circular cross section. It would be apparent that various other cross-sections can be obtained by selecting a package of appropriate nozzle shape such as square, rectangle, hexagon and oval shape.

FIG. 1 shows an embodiment in which the emulsion phase forms a circular core E, which is surrounded by the shell of the gel phase G. FIG. 2 shows another embodiment of a core-shell type configuration where the circular core E extends further towards the periphery of the circle defined by the gel phase G. FIG. 3 and 5 represents another embodiment of a core-shell type configuration where the core of the emulsion phase E is star shaped or cross-shaped in cross-section respectively. FIG. 6 represents an embodiment in which the oval shaped core of the emulsion phase E is offset or eccentrically located within the gel phase G. FIG. 8 represents an embodiment in which the core of emulsion phases E is shaped in the form of a band.

It is also possible to have more than one distinct region of the emulsion or gel phases in the cosmetic composition. FIG. 4 shows such an embodiment having diamond shaped regions E1 E2, E3 and E4 of the emulsion phase within the gel phase G. Alternative such embodiments are depicted in FIGS. 7 and 9 having respectively multiple hexagonal regions E1, E2, E3, E4, E5 and E6 within gel phase G and multiple streaks E1, E2, E3 and E4 within gel phase G.

Gel Phase

Gel-based cosmetics are known in the art. They are generally formulated using an appropriate base, hydrophilic or hydrophobic, such as water or oil, and by adding suitable gel-forming materials, commonly referred to as thickeners. Gels are generally formulated as low solids compositions as they contain a high proportion of the base material. Hydrophilic gels usually produce an intense cooling sensation on the skin and provide a high degree of moisturization. They can also be used to deliver high levels of hydrophilic actives. Hydrophobic gels can be used as delivery vehicles for oil-soluble actives.

The gel phase used for this invention can be hydrophilic or hydrophobic in nature, but is preferably hydrophilic.

The gel phase can comprise from about 50 to 90%, preferably 60 to 95% and more preferably 80 to 90% by weight carrier.

A hydrophilic gel phase is defined for the purpose of this invention as a gel wherein the carrier is hydrophilic. The carrier is preferably selected from one of more of water, ethyl alcohol and isopropyl alcohol. The most preferred carrier is water.

“Hydrophobic gels” for the purpose of this invention are defined as gels in which the carrier is hydrophobic in nature. Preferred hydrophobic carriers are oils, thickened oils, silicone oils and mixtures thereof.

The gel phase has at least one thickening or gelling agent which can be selected from the group consisting of starch, modified starches, cellulose gum, modified cellulose gums, xanthan gum, modified xanthan gums, guar gum, modified guar gums, inorganic thickeners, silica based thickeners, acrylic polymers and mixtures thereof. It is preferable to have inorganic thickeners as the thickening agent. The thickening or gelling agent is present from 0.01 to 10%, preferably 0.5 to 5% and more preferably 0.5 to 1% by weight of the gel phase. High levels of thickeners may lead to a sticky and tacky gel and may also adversely affect its transparency.

The gel phase can be transparent or translucent. It can contain one or more agents selected from silica and polyols (such as glycerine, sorbitol and/or propylene glycol). It is preferable to have a transparent gel phase.

The gel phase can also be suitably colored by incorporating coloring agents.

Optionally the gel phase can have cosmetic adjunct materials such as emollients, preservatives, perfumes, rheology modifiers, humectants, moisturizing agents, skin lightening agents and others generally known in the art.

The viscosity of the gel phase can range from 0.4 to 1.0, preferably 0.6 to 0.9 and more preferably from 0.7 to 0.8 Pa·s at a shear rate of 1000 per second and a temperature of 20° C.

Emulsion Phase

Emulsions are heterogeneous systems of liquids such as oil and water and are generally binary, i.e. two component systems. In these systems, droplets of one liquid are homogenized and stabilized into the other using surface-active agents, which are also called “emulsifiers”. These agents are added to reduce the interfacial tension between the two phases. Emulsions are generally of two types, oil-in-water and water-in-oil. As the name suggests oil-in-water emulsions are ones in which a continuous aqueous phase has droplets of oil/oily material dispersed therein. Water-in-oil emulsions, on the other hand, have a continuous phase of oil/oily material with droplets of an aqueous medium dispersed and stabilized therein.

“Vanishing creams” and “moisturizing creams” are two of the several classes of oil-in-water emulsions that are commonly used in cosmetics. As the name suggests, vanishing creams are designed to spread easily on the skin and then rapidly vanish into it. Such creams are formulated using oils that melt above the normal temperature of the body. These creams are also an effective vehicle for delivery of benefit agents onto the skin and are used to provide a soft matty and draggy feel while and after application. “Cold-creams” form one sub-class of water-in-oil emulsions, used in cosmetics. As the name suggests, these types of emulsions are intended to be used during winter. These creams prevent the skin from becoming dry, patchy and flaky during winter by depositing a thin layer of oil on the skin.

The emulsion phase of the present invention can be an oil-in-water or water-in-oil emulsion, preferably an oil-in-water emulsion. The oil-in-water emulsion preferably has 20 to 85% by weight water, 10 to 70% by weight oil and 0.1 to 5% by weight emulsifier. The term oil includes oils such as castor oil, coconut oil, soyabean oil, olive oil, thickened oils and oily substances such as long chain (C₈-C₂₀) fatty acids and alcohols.

It is preferred that the oil phase comprises mono carboxylic fatty acids with a carbon number from C₈ to C₂₂, preferably C₁₀ to C₁₈ and more preferably from C₁₄ to C₁₈. It is highly preferred that from about 10% to 25% of the total fatty acid is neutralized with a suitable base such as sodium hydroxide or potassium hydroxide. Such compositions are referred to in the art as “vanishing creams”.

The emulsion phase can be advantageously used to provide skin lightening benefits by including suitable skin lightening agents including, for example, one or more of niacinamide, kojic acid, 4-ethyl resorcinol, hydroquinone, tridecyl salicylate and others known in the art. The amount of skin lightening agent can range from 0.01 to 5%, preferably 0.1 to 3%, and more preferably 0.5 to 2% by weight of the emulsion phase. Niacinamide is the most preferred skin lightening agent.

The viscosity of the emulsion phase can range from 0.1 to 0.3, preferably 0.15 to 0.25 and more preferably 0.2 Pa·s at a shear rate of 1000 per second and a temperature of 20° C.

The emulsifier is selected from the group of anionic, cationic, nonionic, amphoteric and zwitterionic surfactants generally known in the art. It is preferred that the emulsifier is an anionic surfactant and more preferably a soap, that is, sodium or potassium salt of fatty acid.

Common Ingredients

The individual phases may additionally comprise materials commonly employed in skin care products such as liquid or solid emollients, silicone oils, emulsifiers, solvents, humectants, polymeric or inorganic thickeners, powders, organic or inorganic sunscreens, skin lightening agents, skin conditioners, optical brighteners, propellants, healing agents (e.g. allantoin), cooling agents (e.g. urea, menthol, menthyl lactate, frescolate), antiseptic agents and other specific skin-benefit actives. The vehicle may also further include adjuncts such as antioxidants, perfumes, opacifiers, preservatives, colorants and buffers.

A safe and effective amount of sunscreen may be used in the compositions of the subject invention. The composition preferably comprises from 0.1% to 10%, more preferably from 0.1% to 5% by weight of the composition of a sunscreen agent. The sunscreen agent may comprise either organic or inorganic sunscreen or combinations thereof. The organic sunscreens are preferably chosen from 4-tertiary butyl-4′-methoxy dibenzoylmethane, available under the trade name PARSOL™-1789 (Givaudan) or 2-ethyl hexyl methoxy cinnamate, available under the trade name PARSOL™-MCX (Givaudan) or mixtures of the two sunscreen compounds. Inorganic sunscreens, which may be employed, include titanium dioxide, zinc oxide or silica (such as fumed silica) and mixtures thereof. These inorganic sunscreens are preferably in micronized form. Ultrafine titanium dioxide in either of its two forms, namely water-dispersible titanium dioxide and oil-dispersible titanium dioxide, is particularly suitable for the invention. Water-dispersible titanium dioxide is ultra-fine titanium dioxide, the particles of which are non-coated or which are coated with a material to impart a hydrophilic surface property to the particles. Examples of such materials include aluminium oxide and aluminium silicate. Oil-dispersible titanium dioxide is ultrafine titanium dioxide, the particles of which exhibit a hydrophobic surface property, and which, for this purpose, can be coated with metal soaps such as aluminium stearate, aluminium laurate or zinc stearate, or with organosilicone compounds. By “ultrafine or micronized form” is meant particles of inorganic sunscreens having an average particle size of less than 100 nm, preferably 70 nm or less, more preferably less than 40 nm and most preferably from 15 nm to 25 nm.

Vitamins, which act as skin-lightening ingredients can be advantageously included in the composition to provide for additional skin lightening effects. These include vitamin B6, vitamin C, vitamin A or their precursors and cosmetically acceptable derivatives. Mixtures of the vitamins can also be employed in the composition of the invention. When present, these vitamins are used in the range of 0.01 to 10.0% by weight of said composition.

Emollients which may be employed include one or more of stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palpitate, silicone oils (such as dimethylpolysiloxane), di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate and myristyl myristate.

Propellants may also be employed with the composition and include propane, butane, isobutane, dimethyl ether, carbon dioxide, nitrous oxide and mixtures thereof.

Solvents include ethyl alcohol, isopropanol, acetone/ethylene glycol monoethyl ether, diethylene glycol monobutyl ether, diethylene glycol monoethyl ether and mixtures thereof.

Powders include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate and mixtures thereof.

Plant extracts include those from genus rubia, symplocus, curcuma and mixtures thereof.

Perfume/fragrance ingredients may also be included in the composition at ranges from 0.001 to 40.0% by weight of the composition.

Product Packaging and Use

The term “topical cosmetic composition” as used throughout the specification means cosmetics which are intended to be used on the skin, which includes the face, neck region, legs and hands. More preferably the product is a leave-on product (a product to be applied to the skin without a deliberate rinsing step soon after its application to the skin).

The composition can be packed in suitable squeezable tubular packs with a dispensing opening (nozzle) through which the product can be extruded. Alternatively the product can be packed in hand actuated dispensing pumps or flexible pouches with a closure. Such packs are generally known in the art as form-fill-seal packages.

The invention will now be described with the help of the following examples.

### EXAMPLE 1

A core and sheath type of cosmetic composition as depicted in FIG. 2 was prepared as described hereunder by using ingredients listed in table 1 below.

Formulation of Gel Phase

0.6 g Aristoflex™ and 0.25 g Laponite™ were dispersed in 20 ml water under overhead stirring. 0.6 g glycerin and 2.5 g propylene glycol were added to this dispersion. Preservatives were dissolved in 10 ml water and this solution was added to the dispersion. Niacinamide and urea were dissolved in separate portions of water (7 ml each) and added to the above dispersion. The mixture was stirred and deaerated under vacuum to remove entrapped bubbles of air.

Formulation of Emulsion Phase

Glycerin, niacinamide and potassium hydroxide were mixed with water and this solution was heated to 80° C. In another container, the oil-soluble ingredients (stearic acid, oil-soluble preservatives, cetyl alcohol, DC-200™, etc) were heated to 80° C. The oil phase was then added to the water phase to form an oil-water phase. Parsol™ MCX and Parsol™ 1789 were then warmed and titanium dioxide (MT 100Z™—size 15 nm, supplied by Tayka Corporation, Japan) dispersed therein to form a sunscreen mix. The sunscreen mix was then added to the above oil-water phase. Perfume and coloring matter were added finally.

Filling Operation

The composition was filled into tubes using a co-extrusion machine. The gel phase and cream phase were stored in separate pots, which were each connected to the filling nozzle of the co-extrusion machine. The nozzle diameter was chosen such that the tubes fitted snugly into it. The cohesive value, viscosity, bulk density of the gel and emulsion as well as the pump setting and flow rate were suitably adjusted such that the two phases co-extruded as desired and were filled in the tubes after proper weight adjustment.

TABLE 1

| Ingredients (% w/w) | Cream | Gel | Total |
|---|---|---|---|
| Water | 37.00 | 44.00 | 81.00 |
| Glycerin | 0.5 | 0.67 | 1.17 |
| Niaciamide | 0.5 | 0.67 | 1.17 |
| Phenoxyethanol | 0.1 | 0.13 | 0.23 |
| Potassium Hydroxide | 0.29 | 0 | 0.29 |
| Stearic acid | 8.95 | 0 | 8.95 |
| Cetyl alcohol | 0.27 | 0 | 0.27 |
| DC 200 ™⁽¹⁾ | 0.25 | 0 | 0.25 |
| Isopropyl myristate | 0.38 | 0 | 0.38 |
| Parsol ™ MCX⁽²⁾ | 0.38 | 0 | 0.38 |
| Parsol ™ 1789⁽³⁾ | 0.2 | 0 | 0.2 |
| Aristoflex ™ AVC⁽⁴⁾ | 0 | 0.6 | 0.6 |
| Laponite ™ XLG⁽⁵⁾ | 0 | 0.25 | 0.25 |
| Propylene glycol | 0 | 2.5 | 2.5 |
| Perfume | 0.3 | 0 | 0.3 |
| Balance of minor | 50.00 | 50.00 | 100.00 |
| ingredients to | | | |

⁽¹⁾Polydimethyl Siloxane fluid (350 cSt), supplied by the Dow Corning Corporation.
⁽²⁾2-Ethyl hexyl methoxy cinnamate, supplied by Givaudan.
⁽³⁾4-Tertiary butyl-4′-methoxy dibenzoylmethane, supplied by Givaudan.
⁽⁴⁾Acryloyldimethyltaurate copolymer, supplied by the Clariant Corporation.
⁽⁵⁾Synthetic clay-based inorganic thickener from Southern Clay Products, Inc.

Storage Stability Testing

Samples of the above composition filled in tubes as described above were stored at 45° C. and room temperature (˜20° C.) for six weeks. After this storage it was observed that the gel and emulsion phases still retained their gloss and overall stability (integrity on visual observation).

### EXAMPLE 2

A core and sheath type of cosmetic composition as depicted in FIG. 2 was prepared by using the ingredients listed in table 2 in a similar way to that used for the preparation of the composition of example 1. The gel phase and the cream phase were present in amounts of 50% and 50% with respect to the weight of composition.

TABLE 2

| | |
|---|---|
| Ingredient | Amount (% w/w) |

Gel phase:

| | |
|---|---|
| Water | 78.4 |
| Propylene glycol | 8.0 |
| Glycerine | 5.0 |
| Niacinamide | 5.0 |
| Aristoflex AVC | 1.4 |
| Laponite | 0.4 |
| Allantoin | 0.4 |
| Minors | To 100 |

Cream phase:

| | |
|---|---|
| Water | 70.1 |
| Stearic acid | 15.0 |
| Isopropyl myristate | 2.0 |
| Cetyl alcohol | 1.0 |
| Glycerine | 1.0 |
| Niacinamde | 3.0 |
| Silicone | 1.0 |
| Parsol MCX | 2.0 |
| Parsol 1789 | 1.5 |
| Micronized TiO₂ | 0.8 |
| Allantoin | 0.4 |
| Potassium hydroxide | 0.5 |
| Minors | To 100 |

Minors include colour, preservatives, chelants etc.

An 8 week clinical study was conducted in India on 35 men to assess the skin lightening efficacy of the composition. The study was done on the lateral and volar forearm and the composition was randomized on both of the arms to the same extent. 20 mg of the composition was applied twice daily at least six hours apart, each application consisting of 10 rubs. Hands were washed prior to each application.

The initial skin color of the inner side of their forearms was determined visually by a trained expert and was allotted a score between 1 (very fair) to 10 (very dark). At the end of the trial the change in skin color of the treated sites was recorded again by the same trained expert.

The difference between the initial skin colour score and the final score is indicative of the efficacy of the formulation. A negative value indicates skin lightening and a positive value indicates skin darkening with respect to the initial skin color.

The skin lightening efficacy results were as follows:

| Product | 4 weeks | 6weeks | 8 weeks |
|---|---|---|---|
| Example 2 | −0.1 | −0.16 | −0.16 |

The more negative the skin lightening efficacy, the better the skin lightening efficacy. Statistically significant skin lightening was observed only four weeks after the composition was first applied.

A natural context product test was carried out with 440 consumers in two cities in India over a ten day test. The results of the study indicated significantly better scores on cooling, freshness, fairness, visual appearance, and uniqueness for the composition than a conventional skin lightening cream (Fair & Lovely multivitamin fairness cream available from Hindustan Lever Limited).

An anti-tanning study was also conducted to assess the tan protection index (TPI) {[Grade of tan level on untreated site−Grade of tan level on treated site]/Grade of tan level on untreated site) of the product. The tan protection efficacy is largely a result of the UV-A component of sunscreens. The study was a double blind design in which both study personnel and study subjects did not know which composition was thre test composition. Both forearms (area from elbow to wrist) were were used as treatment sites. Sites were exposed to the mid-day sun for 30 minutes and clinically/visually evaluated prior to application and immediately after exposure to the sun on the first day of the study. The evaluation was performed by expert graders using a tanning scale from 0 (no difference) to 8 (extremely dark with peeling). The application and sun exposure continued for 3 consecutive days. As melanogenesis usually peaks around 5-7 days following exposure, the sites were visually evaluated for tan up to 10 days. On the tenth day, the TPI was calculated. The greater the TPI value, the better the tan protection (i.e, less tanning is observed).

The results are as follows:

| Product | TPI day 5 | TPI day 8 | TPI day 10 |
|---|---|---|---|
| Example 2 | 51 | 52 | 57 |

The composition showed statistically significant anti-tanning efficacy on day 8 and 10.

1. A topical multiphase cosmetic composition comprising an emulsion phase and a gel phase, said phases being co-extensive and having at least one common interface, wherein said phases are disposed such that the gel phase forms greater than 50% of the outer surface of the composition.

2. The topical multiphase cosmetic composition as claimed in claim 1 wherein the emulsion phase is an oil-in-water emulsion and the gel phase is a hydrophilic gel.

3. A topical multiphase cosmetic composition as claimed in claim 1 wherein the gel phase forms greater than 80% of the outer surface of the composition.

4. A topical multiphase cosmetic composition as claimed in claim 1 wherein the gel phase forms the entire outer surface of the composition.

5. A topical multiphase cosmetic composition as claimed in claim 1 wherein the emulsion phase and the gel phase are coaxially disposed.

6. A topical multiphase cosmetic composition as claimed in claim 1 wherein the emulsion phase and the gel phase are present in a % volume ratio of 10:90 to 90:10 of the composition.

7. A topical multiphase cosmetic composition as claimed in claim 6 wherein the % volume ratio is 40:60 to 60:40 of the composition.

8. A topical multiphase cosmetic composition as claimed in claim 1 wherein the gel phase comprises:
(a) 50 to 90% by weight carrier selected from the group consisting of water, ethyl alcohol, isopropyl alcohol and combinations thereof; and
(b) 0.01 to 10% by weight thickening agent.

9. A topical multiphase cosmetic composition as claimed in claim 8 wherein the carrier is water.

10. A topical multiphase cosmetic composition as claimed in claim 1 wherein the emulsion phase comprises:
(A) 20 to 85% by weight water;
(B) 10 to 70% by weight oil; and
(C) 0.1 to 5% by weight emulsifier.
